(19) United States
(12) Patent Application Publication
(10) Pub. No.: US 2025/0230438 A1
(43) Pub. Date: Jul. 17, 2025

(54) NEURAL REPAIR THROUGH INHIBITION OF REG3A SIGNALING

(71) Applicant: UNIVERSITY OF SOUTH CAROLINA, COLUMBIA, SC (US)

(72) Inventors: JEFFERY TWISS, COLUMBIA, SC (US); COURTNEY N. BUCHANAN, COLUMBIA, SC (US); JINYOUNG LEE, COLUMBIA, SC (US); LAUREN VAUGHN, IRMO, SC (US); ASHLEY KALINSKI, Yorktown, IN (US)

(21) Appl. No.: 18/941,451

(22) Filed: Nov. 8, 2024

Related U.S. Application Data
(60) Provisional application No. 63/597,149, filed on Nov. 8, 2023.

(51) Int. Cl. C12N 15/113; A61P 25/00; G01N 33/68

(52) U.S. Cl. CPC C12N 15/113; A61P 25/00; G01N 33/6896; C12N 2310/531; C12N 2320/35; G01N 2800/28

(57) ABSTRACT

In general, disclosed herein are methods for increasing nerve regeneration. The method includes, for instance, administering an effective amount of an agent comprising a sequence at least 95% identical to SEQ ID NOs: 1-3 to a subject, wherein the agent depletes or inhibits Reg3A or KHSRP expression in neurons of the subject.

### CROSS REFERENCE TO RELATED APPLICATIONS

This application claims the filing benefit of U.S. Provisional Patent Application Ser. No. 63/597,149, filed on Nov. 8, 2023, which is incorporated herein by reference.

### GOVERNMENT SUPPORT CLAUSE

This invention was made with government support under grant R01 NS089633 awarded by National Institutes of Health (NIH). The government has certain rights in the invention.

### SEQUENCE LISTING

The instant application contains a Sequence Listing which has been submitted electronically in XML format and is hereby incorporated by reference in its entirety. Said XML copy, created on Mar. 28, 2025, is named USC-809_1677_SL.xml and is 3,632 bytes in size.

### BACKGROUND

More than 18,000 hospitalized individuals will have a peripheral nerve injury in the US annually. If one extends this to non-hospitalized individuals with compression injuries (e.g., spinal disc herniation, entrapment neuropathies), needs for regeneration promoting therapies are a huge market. For spinal cord injury, there are similarly about 18,000 new cases annually in the US. Because the mechanisms inhibiting axon regeneration in spinal cord injury also block axon regeneration after stroke and optic nerve injury, the potential market grows substantially.

The RNA binding protein KHSRP is locally synthesized in axons after injury and slows peripheral nerve regeneration (Patel et al., 2022). Translation of axonal Khsrp mRNA is activated by an axotomy-induced increase in axoplasmic calcium that returns to pre-injury levels several hours after axotomy that allows intra-axonal synthesis of growth-promoting proteins. However, axonal KHSRP protein remains elevated for weeks after nerve injury throughout the course of regeneration. Further, an axon-to-axon signaling mechanism that maintains axonal KHSRP protein levels through translation of two mRNAs in axons, Reg3A and Khsrp. Reg3a mRNA is transported into peripheral axons a few days after axotomy, where it is translated and the resulting protein product is secreted from the distal axon. REG3A is a small lectin like protein that has been linked to pancreatic islet cell regeneration and inflammation. In sensory neurons, REG3A activates calcium release from the endoplasmic reticulum, activating PERK to phosphorylate eIF2α, which increases Khsrp mRNA translation.

There are pressing clinical needs to improve regeneration after nerve, spinal cord, and brain injury. Disclosed herein are methods to increase regeneration by targeting a particular signaling pathway that slows regeneration. Blocking this pathway speeds up regeneration and accelerates recovery after traumatic nerve injury. This brings a new strategy to target endogenous mechanisms that decrease successful regeneration.

### SUMMARY

In general, disclosed herein are methods for increasing nerve regeneration. The method includes, for instance, administering an effective amount of an agent comprising a sequence at least 95% identical to SEQ ID NOs: 1-3 to a subject, wherein the agent depletes or inhibits Reg3A or KHSRP expression in neurons of the subject.

Also, disclosed herein are methods for treating a nerve injury. The method includes, for instance, administering an effective amount of an agent comprising a sequence at least 95% identical to SEQ ID NOs: 1-3 to a nerve injury site of a subject, wherein the agent depletes or inhibits Reg3A or KHSRP expression in neurons of the subject.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A depicts representative neurofilament-immunofluorescent images of primary dissociated mouse DRG neurons untreated and after being treated with recombinant REG3A protein (recREG3A). The recREG3A decreases axon extension from primary dissociated mouse DRG neurons.

FIG. 1B depicts quantitation of axon lengths after 24-hour exposure of primary dissociated mouse DRG neurons to recREG3A.

FIG. 1C depicts efficacy for small hairpin RNA targeting Reg3a (shReg3a) in reducing endogenous Reg3a mRNA from axons based on fluorescence in situ hybridization and confocal imaging.

FIG. 1D depicts efficacy for small hairpin RNA targeting Reg3a (shReg3a) depleting endogenous Reg3a mRNA from axons based on immunofluorescence in situ hybridization and confocal imaging.

FIG. 1E depicts depletion of endogenous REG3A increases axon growth in dissociated adult mouse DRG cultures (P values by student's t-test).

FIG. 2A depicts representative immunoblot of protein lysates from uninjured adult mouse CNS (optic nerve, spinal cord, brain) and PNS (sciatic nerve, DRG).

FIG. 2B depicts comparison of uninjured to crush injured sciatic nerve REG3A protein expression.

FIG. 3 depicts representative confocal images of distal axons in adult L4-6 DRG cultures from naïve and 7-day injury-conditioned mice±heparinase.

FIG. 4A depicts Reg3a mRNA from the L4-6 DRGs expression levels measured by reverse transcriptase-coupled polymerase chain reaction (RT-PCR) using droplet digital technology in AAV-shReg3a compared to AAV encoding a non-targeting shRNA (shCntl) transduced neurons.

FIG. 4B depicts representative immunofluorescence images for SCG10 protein as a marker of regeneration in cryostat sections of sciatic nerve from AAV-shCntl vs. AAV-shReg3a transduced mice at 14 days post injury.

FIG. 4C depicts quantitation of SCG10 axon profiles at distances beyond the crush site (regeneration index) at the indicated day post-nerve injury and shows that the AAV-shReg3a transduced mice have accelerated axon regeneration (* p≤0.05, ** p≤ 0.01, *** p≤0.005, and **** p≤0.0001 by repeated measures ANOVA for shReg3a vs. shCntl).

FIG. 5A illustrates a schematic of DRG spot cultures used for measurement of regeneration rates.

FIG. 5B depicts representative image sequences for BFP-filled axons for DRG spot cultures transduced with AAV-shCntl vs. AAV-shReg3a.

FIG. 5C depicts quantitation overall axon regrowth over 4.5 h sequence and change in axon growth every 10 min across the 4.5-hour sequence.

FIG. 6A depicts axon growth after recREG3A treatment with and without cycloheximide (CHX) treatment in dissociated DRG cultures (P values by ANOVA with Tukey post-hoc).

FIG. 6B depicts axon branching after recREG3A treatment with and without cycloheximide (CHX) treatment in dissociated DRG cultures (P values by ANOVA with Tukey post-hoc).

FIG. 6C depicts axonal levels of KHSRP protein after recREG3A treatment with and without cycloheximide (CHX) treatment in DRG cultures.

FIG. 6D depicts representative images sequences for FRAP using GFP^{MYR}5′/3′khsrp reporter for intra-axonal translation.

FIG. 6E depicts quantitation of fluorescent recovery under basal (control), recREG3A and recREG3A+CHX (* p≤0.05, ** p≤0.01, *** p≤0.005, and **** p≤0.001 for indicated lines vs. control [color matches comparing data points] by repeated measures ANOVA).

FIG. 7A depicts percentage of recovery of axonal Reg3a mRNA translation reporter in the absence and presence of the protein synthesis inhibitor cycloheximide (CHX) after photobleaching (* p≤0.05, ** p≤0.01, *** p≤0.005, and **** p≤0.001 for indicated lines vs. control [color matches comparing data points] by repeated measures ANOVA).

FIG. 7B depicts dual FRAP assay for recovery of axonal Reg3a and Khsrp mRNA translation reporters in the same axons simultaneously show that increasing axoplasmic calcium increases recovery of GFP^{MYR}5′/3′khsrp while decreasing recovery of mCherry^{MYR}5′/3′reg3a.

FIG. 7C depicts intracellular calcium chelation using BAPTA-AM that increases recovery of mCherry^{MYR}5′/3′reg3a while decreasing recovery of GFP^{MYR}5′/3′khsrp using a dual FRAP assay.

FIG. 8A depicts the effect of recREG3A on axon growth following chelation of intracellular calcium using BAPTA-AM and extracellular calcium using BAPTA.

FIG. 8B depicts the effect of recREG3A on axon branching following chelation of intracellular calcium using BAPTA-AM and extracellular calcium using BAPTA.

FIG. 8C depicts axonal KHSRP protein following intracellular and extracellular calcium chelation (P values by ANOVA with Tukey post-hoc analyses in FIGS. 8A-8C).

FIG. 8D depicts axonal calcium levels based on GCaMP fluorescence following recREG3A treatment vs. control and depletion of REG3A using AAV-shReg3a decreases basal calcium levels vs. AAV-shCntl transduced DRG cultures. RecREG3A increases while REG3A depletion decreases intra-axonal calcium (P values by student's t-test).

FIG. 8E depicts time lapse imaging for growth calcium levels based on GCaMP fluorescence and axon growth events over time is shown for AAV-shCntl vs. AAV-shReg3a transduced DRG spot cultures beginning at 16 hours post axotomy.

FIG. 9A depicts REG3A-dependent axon growth following pharmacological inhibition of PERK in dissociated DRG cultures (P values by 2-way ANOVA with Tukey's post-hoc test).

FIG. 9B depicts axonal KHSRP protein levels following pharmacological inhibition of PERK (P values by 2-way ANOVA with Tukey's post-hoc test).

FIG. 9C depicts FRAP assays to test for axonal translation of Khsrp mRNA reporter following pharmacological inhibition of PERK in dissociated DRG cultures.

FIG. 9D depicts representative images following recREG3A treatment of dissociated DRG cultures (P values by 2-way ANOVA with Tukey post-hoc analysis).

FIG. 9E depicts quantitative immunofluorescent imaging following recREG3A treatment of dissociated DRG cultures (P values by 2-way ANOVA with Tukey post-hoc analysis).

FIG. 10A illustrates mCherry^{MYR}5′/3′reg3a and GFP^{MYR}5′/3′khsrp translation reporter plasmids.

FIG. 10B depicts quantitation of mCherry^{MYR} (Reg3a mRNA translation reporter) and GFP^{MYR} (Khsrp mRNA translation reporter) fluorescence in distal axons over time beginning at 16 hours post axotomy in DRG spot cultures.

Repeat use of reference characters in the present specification and drawings is intended to represent the same or analogous features or elements of the present invention.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment may be used in another embodiment to yield a still further embodiment. Thus, it is intended that the present disclosure covers such modifications and variations as come within the scope of the appended claims and their equivalents.

Current approaches for peripheral nerve injuries revolve around surgical interventions to place conduits that support regeneration or exogenous electrical stimulation to improve axon growth. Human peripheral nerve regeneration is rarely successful when the nerve's axons have to regrow more than 5-6 cm. By the time the regenerating axons in the nerve get past that 5-6 cm, the environment no longer supports growth and target tissues are no longer receptive for reinnervation. Advantageously, methods disclosed herein are directed to increasing nerve regeneration by selectively inhibiting intra-neuronal (e.g., intra-axonal) signaling pathways that are increased during regeneration thereby slowing down axon growth rates. By inhibiting the pathways that Reg3A activates in axons, axon growth may be accelerated above the typical 1-2 mm/day. Consequently, axons can reach their targets before the environment loses growth support and target tissues lose receptiveness for reinnervation.

In some example embodiments, methods for increasing nerve regeneration may include administering to a subject having, or suspected of having, a nerve injury an agent that completely or at least partially depletes, inhibits, prevents, or modulates Reg3A and/or KHSRP. Reg3A protein is synthesized in and released from regenerating axons. The secreted Reg3A protein activates a signaling pathway that converges on KHSRP, to promote decay of mRNAs in axons. Deletion of Reg3A or KHSRP from neurons accelerates peripheral nerve regeneration. Reg3A mRNA appears to only localize into regenerating axons, so this provides an autocrine mechanism that neurons use to slow their rate of growth. Interfering with axonal localization of Reg3A mRNA, intra-axonal translation of Reg3A mRNA or the signals downstream of Reg3A protein binding to its cell surface receptor bring opportunities to accelerate peripheral nerve regeneration. In one example embodiment, methods for increasing nerve regeneration may include administering to a subject having, or suspected of having, a nerve injury an agent that completely or at least partially depletes, inhibits, prevents, or modulates Reg3A. In another example embodiment, methods for increasing nerve regeneration may include administering to a subject having, or suspected of having, a nerve injury an agent that completely or at least partially depletes, inhibits, prevents, or modulates KHSRP.

In some example embodiments, the agent that completely or at least partially depletes, inhibits, prevents, or modulates Reg3A and/or KHSRP may include, but is not limited to, a small molecule inhibitor, a small molecule degrader, a CRISPR guide RNA (gRNA), an RNA interfering agent, an oligonucleotide, a peptide or peptidomimetic inhibitor, an aptamer, an antibody, or an intrabody. In one example embodiment, for instance, the agent may be a small molecule inhibitor. In another example embodiment, for instance, the agent may be an RNA interfering agent. In yet another example embodiment, for instance, the agent may be an oligonucleotide.

In one example embodiment, the agent disclosed herein may depletes levels of Reg3A or KHSRP by at least 70% or more as compared to a control sample. In one example embodiment, the agent disclosed herein may depletes levels of Reg3A or KHSRP by at least 90% or more as compared to a control sample.

For instance, the agent (such as, shRNAs) may be incorporated into a protein complex that recognizes and cleaves target Reg3A mRNA or KHSRP mRNA. RNAi may also be initiated by introducing shReg3A to neurons of a subject to inhibit or deplete the expression of Reg3A or KHSRP. As used herein, “depletion” or “inhibition” of Reg3A or KHSRP includes any decrease in expression, protein activity, or level of the target biomarker. In one example embodiment, shReg3A may depletes levels of Reg3A or KHSRP by at least 30%, 40%, 50%, 60%, 70%, 80%, 90%, 95%, or 99% or more as compared to the expression of a target biomarker nucleic acid or the activity or level of the protein encoded by a target biomarker nucleic acid which has not been targeted by an RNA interfering agent.

In one example embodiment, shReg3A may deplete levels of Reg3A or KHSRP by at least 70% or more as compared to a control sample. In one example embodiment, shReg3A may deplete levels of Reg3A or KHSRP by at least 90% or more as compared to a control sample.

In one example embodiment, for instance, the vector may be a DNA construct or a viral vector. In one example embodiment, the DNA construct may be delivered to tissues of a subject by transfection, i.e., by delivery of “naked” DNA or in a complex with a colloidal dispersion system. A colloidal system may include, but is not limited to, macromolecule complexes, nanocapsules, microspheres, beads, and lipid-based systems including oil-in-water emulsions, micelles, mixed micelles, and liposomes.

In one example embodiment, the agent may be an RNA interfering agent. As used herein, an “RNA interfering agent” refers to an agent that interferes with or inhibits expression of a target biomarker gene by RNA interference (RNAi). Methods for constructing RNA interfering agents are well known in the art. For example, the interfering RNA may be assembled from two separate oligonucleotides, in which one strand is the sense strand and the other is the antisense strand, wherein the sense and antisense strands are self-complementary (i.e., each strand comprises nucleotide sequence that is complementary to nucleotide sequence in the other strand; such as where the antisense strand and sense strand form a duplex or double stranded structure); the antisense strand includes a nucleotide sequence that is complementary to a nucleotide sequence in a target nucleic acid molecule or a portion thereof (i.e., an undesired gene) and the sense strand includes nucleotide sequence corresponding to the target nucleic acid sequence or a portion thereof. Alternatively, interfering RNA may be assembled from a single oligonucleotide, where the self-complementary sense and antisense regions are linked by means of nucleic acid based or non-nucleic acid-based linker(s). The interfering RNA may be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering may be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide may be processed either in vivo or in vitro to generate an active siRNA molecule capable of mediating RNA interference.

In one example embodiment, the RNA interfering agent may include, but is not limited to, a small interfering RNA (siRNA), CRISPR RNA (crRNA), CRISPR guide RNA (gRNA), a small hairpin RNA (shRNA), a microRNA (miRNA), or a piwi-interacting RNA (piRNA). For instance, in one example embodiment, the RNA interfering agent may be a shRNA. The loop region may generally be at least about 2 nucleotides in length, such as at least about 5 nucleotides in length. The loop region may generally be less than about 10 nucleotides in length, such as less than about 8 nucleotides in length. The sense region and the antisense region may be between about 12 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

In some example embodiments, the agent disclosed herein may be delivered to the neurons of a subject via a transporter molecule. For instance, a nucleic acid sequence of the agent may be inserted into delivery vectors and expressed from transcription units within the vectors. Nucleic acids may be delivered in any desired vector. These may include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids may be administered in any desired format that provides sufficiently efficient delivery levels, including in virus particles, in liposomes, in nanoparticles, and complexed to polymers. Vector construct may be produced using any suitable genetic engineering techniques well known in the art, including, without limitation, the standard techniques of PCR, oligonucleotide synthesis, restriction endonuclease digestion, ligation, transformation, plasmid purification, and DNA sequencing. The selection of an appropriate vector to deliver nucleic acids and optimization of the conditions for insertion of the selected expression vector into a cell, are within the scope of one of ordinary skill in the art without the need for undue experimentation.

The target RNA cleavage reaction guided by shRNAs is highly sequence specific. As such, the shRNA herein includes nucleotide sequences identical to a portion of the nucleic acid that is being targeted for depletion or inhibition. However, 100% sequence identity between the shRNA and the target gene is not required. Thus, the methods disclosed herein may be able to tolerate sequence variations that might be expected due to genetic mutation, strain polymorphism, or evolutionary divergence. For instance, shRNA sequences with insertions, deletions, and single point mutations relative to the target sequence have also been found to be effective for depletion or inhibition. In general, the shRNAs retain specificity for their target, i.e., must not directly bind to, or directly significantly affect expression levels of, transcripts other than the intended target. In one example embodiment, the shRNA sequence may be at least about 60%, 65%, 70%, 75%, 80%, 85%, 90%, 91%, 92%, 93%, 94%, 95%, 96%, 97%, 98%, or 99% identical to the messenger RNA (mRNA) sequence of the targeted gene over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases. In one example embodiment, for instance, the shRNA sequence may be at least about 95%, 96%, 97%, 98%, or 99% identical to the mRNA sequence of the targeted gene over a region of about 5, 10, 15, 20, 25, 30, or more nucleobases. In another example embodiment, for instance, the shRNA sequence may be at least about 98% identical to the mRNA sequence of the targeted gene over a region of about 5, 10, 15, 20, 25, 30, or more nucleobases.

TABLE 1
Sequences

| SEQ ID NO: | SEQUENCE |
|---|---|
| 1 | gatccaacaatgggtcaacaa |
| 2 | DPTMGQQ |
| 3 | DPTQGTE |

In one example embodiment, the agent administered to a subject having or suspected of having, a nerve injury may be a sequence presented in Table 1. For instance, in one example embodiment, the agent may be a sequence at least 95% identical to SEQ ID NO: 1 (5′-GATCCAACAATGGGTCAACAA-3′). In another example embodiment, the agent may be a sequence at least 95% identical to SEQ ID NO: 2 (DPTMGQQ). In another example embodiment, the agent may be a sequence at least 95% identical to SEQ ID NO: 3 (DPTQGTE).

In one example embodiment, the agent administered to a subject having or suspected of having, a nerve injury may be an shRNA. For instance, in one example embodiment, the shRNA (e.g., shReg3A) may be SEQ ID NO: 1 (5′-GATCCAACAATGGGTCAACAA-3′).

In one example embodiment, the shRNA may be administered to a subject having or at risk for having a nerve injury, to deplete or inhibit expression of Reg3A or KHSRP, which are overexpressed in the nervous system during a nerve injury, and thereby increasing nerve regeneration.

The term “subject” refers to any organism to which aspects of the disclosure may be administered, e.g., for experimental, diagnostic, prophylactic, and/or therapeutic purposes. Subjects to which embodiments of the disclosure may be administered include mammals, such as primates and humans. For veterinary applications, a wide variety of subjects are suitable, e.g., livestock such as cattle, sheep, goats, cows, swine, and the like; poultry such as chickens, ducks, geese, turkeys, and the like; and domesticated animals, such as pets such as dogs and cats. For diagnostic or research applications, a wide variety of mammals are suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

In one example embodiment, the agent disclosed herein may be incorporated into a pharmaceutical composition and administered to a subject. For instance, the administration thereof may be carried out in any convenient manner, including aerosol inhalation, injection, ingestion, transfusion, implantation or transplantation. For instance, the agent may be administered orally, subcutaneously, intravenously, intraparenchymal, intrathecal, intra-nerve, intra-brain, intra-spinal cord, or intratumoral. In this regard, “oral” administration can refer to administration into a subject's mouth; “subcutaneous” administration can refer to administration just below the skin; “intravenous” administration can refer to administration into a vein of a subject; and “intrathecal” administration can refer to administration within administration into the cerebrospinal fluid; and ‘intra-nerve or -brain/-spinal cord parenchyma’ can refer to direct injection into the nervous system.

Pharmaceutical compositions disclosed herein may be formulated to be compatible with its intended route of administration. As used herein, “routes of administration” may include, but are not limited to, parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition may be sterile and should be fluid to the extent that easy syringability exists. It may be stable under the conditions of manufacture and storage and must be preserved against the contaminating action of microorganisms such as bacteria and fungi. The carrier may be a solvent or dispersion medium containing, for example, water, ethanol, a pharmaceutically acceptable polyol like glycerol, propylene glycol, liquid polyethylene glycol, and suitable mixtures thereof. Prolonged absorption of the injectable compositions may be brought about by including in the composition an agent which delays absorption, for example, aluminum monostearate and gelatin.

Oral compositions may include an inert diluent or an edible carrier. They may be enclosed in gelatin capsules or compressed into tablets. For the purpose of oral therapeutic administration, the active compound may be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, sodium starch glycolate, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Compositions for parenteral delivery, e.g., via injection, can include pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., corn oil) and injectable organic esters such as ethyl oleate. In addition, the composition can contain minor amounts of auxiliary substances such as wetting or emulsifying agents, pH buffering agents and the like that can enhance the effectiveness of the phenolic compound. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents, and dispersing agents.

In one example embodiment, an effective amount of the agent may be administered to the subject. For instance, an effective amount of the agent may be administered a nerve injury site of a subject, The term “effective amount” refers to those amounts that, when administered to a subject in view of the nature and severity of that subject's nerve injury, will have a desired therapeutic effect, e.g., an amount which will cure, prevent, inhibit, or at least partially arrest or partially prevent a target disease or condition. An effective dose further can refer to that amount of the agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

An effective dose can depend upon a number of factors known to those of ordinary skill in the art. The dosage can vary depending upon known factors such as the pharmacodynamic characteristics of the active ingredient and its mode and route of administration; time of administration of active ingredient; identity, size, condition, age, sex, health and weight of the subject or sample being treated; nature and extent of symptoms; kind of concurrent treatment, frequency of treatment and the effect desired; and rate of excretion. These amounts may be readily determined by the skilled artisan.

In one example embodiment, an effective amount of the agent may be in the range of about 0.005 mg/kg body weight to about 100 mg/kg body weight, such as from about 0.01 mg/kg body weight to about 50 mg/kg body weight, such as from about 0.1 mg/kg body weight to about 30 mg/kg body weight, such as from about 1 mg/kg body weight to about 20 mg/kg body weight, or any range therebetween. The skilled artisan should appreciate that certain factors may influence the dosage required to effectively treat a subject, including but not limited to the severity of the nerve injury, previous treatments, the general health and/or age of the subject, and other diseases present. Moreover, treatment of a subject with an effective amount of an agent disclosed herein may include a single treatment or, preferably, can include a series of treatments. For example, a subject may be treated with an agent disclosed herein in the range of between about 0.1 mg/kg body weight to 30 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of the agent used for treatment may increase or decrease over the course of a particular treatment. Changes in dosage may result from the results of diagnostic assays.

In some example embodiments, Reg3A or KHSRP expression level may be measured prior to and/or subsequent to administering the agent disclosed herein to a subject. For instance, a biological sample may be obtained from a subject to measure Reg3A or KHSRP expression level compared to a control sample. The biological sample from the subject may be from a tissue, such as central nervous system (CNS) or peripheral nervous system (PNS) tissues. The control sample may be from the same subject or from a different subject. The control sample may be a normal, non-diseased sample. However, in some embodiments, such as for staging of disease or for evaluating the efficacy of treatment, the control sample may be from a diseased tissue. The control sample may be a combination of samples from several different subjects.

In one example embodiment, the amounts determined and/or compared in a method described herein may be based on relative measurements, such as ratios (e.g., Reg3A or KHSRP copy numbers, level, and/or activity before a treatment versus after a treatment, such as Reg3A or KHSRP measurements relative to a spiked or man-made control, such Reg3A or KHSRP measurements relative to the expression of a housekeeping gene, and the like). For instance, the relative analysis may be based on the ratio of pre-treatment Reg3A or KHSRP measurement as compared to post-treatment Reg3A or KHSRP measurement. Pre-treatment Reg3A or KHSRP measurement may be made at any time prior to the administration of an agent disclosed herein. Post-treatment Reg3A or KHSRP measurement may be made at any time after the administration of an agent disclosed herein. In one example embodiment, post-treatment Reg3A or KHSRP measurements may be made 1, 3, 5, 7, 10, 14, 21 days or more after administration of an agent disclosed herein. In one example embodiment, post-treatment Reg3A or KHSRP measurements may be made 1 day after administration of an agent disclosed herein. In another example embodiment, post-treatment Reg3A or KHSRP measurements may be made 3 days after administration of an agent disclosed herein. In another example embodiment, post-treatment Reg3A or KHSRP measurements may be made 7 days after administration of an agent disclosed herein.

The pre-determined Reg3A or KHSRP amount and/or activity measurement(s) may be any suitable standard. For example, the pre-determined Reg3A or KHSRP amount and/or activity measurement(s) may be obtained from the same or a different subject for whom a patient selection is being assessed. For instance, the pre-determined biomarker amount and/or activity measurement(s) may be obtained from a previous assessment of the same subject. In such a manner, the progress of the selection of the subject may be monitored over time. In addition, the control may be obtained from an assessment of another subject or multiple subjects, e.g., selected groups of mice, if the subject is a mice.

In one example embodiment, following administration of an agent disclosed herein, the change of Reg3A or KHSRP amount and/or activity measurement(s) from the pre-determined level may be about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. In one example embodiment, the change of Reg3A or KHSRP amount and/or activity measurement(s) from the pre-determined level may be about 2.0-fold or greater. In another example embodiment, the change of Reg3A or KHSRP amount and/or activity measurement(s) from the pre-determined level may be about 3.0-fold or greater. In another example embodiment, the change of Reg3A or KHSRP amount and/or activity measurement(s) from the pre-determined level may be about 4.0-fold or greater. In another example embodiment, the change of Reg3A or KHSRP amount and/or activity measurement(s) from the pre-determined level may be about 5.0-fold or greater.

In one example embodiment, the following administration of an agent disclosed herein, the amount of Reg3A or KHSRP present in a subject's biological sample may be at least about 2 times (×) less compared to a control biological sample, such as at least about 4× less, such as at least about 5× less, such as at least about 10× less, such as at least about 20× less compared to a control biological sample.

Biological samples may be collected from a variety of sources from a subject including, but not limited to, a body fluid sample, tissue sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In one example embodiment, the subject and/or control sample may be selected from the group consisting of tissues, cells, cell lines, histological slides, paraffin embedded tissues, biopsies, whole blood, nipple aspirate, serum, plasma, buccal scrape, saliva, cerebrospinal fluid, urine, stool, and bone marrow. In one example embodiment, the sample may be tissues, cells, serum, plasma, or urine. For instance, tissue samples may include, but are not limited to, brain, optic nerve, sciatic nerve, or spinal cord tissue samples. In one example embodiment, the tissue sample may be a brain tissue sample. In another example embodiment, the tissue sample may be an optic nerve tissue sample. In another example embodiment, the tissue sample may be a sciatic nerve tissue sample. In another example embodiment, the tissue sample may be a spinal cord tissue sample.

In one example embodiment, Reg3A or KHSRP gene expression may be measured prior to and/or subsequent to administering the agent disclosed herein to a subject. For instance, gene expression levels may be assessed by any method well-known in the arts for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In one example embodiment, activity of Reg3A or KHSRP genes may be characterized by a measure of gene transcript (e.g. mRNA), by a measure of the quantity of translated protein, or by a measure of gene product activity. For instance, Reg3A or KHSRP expression may be monitored by detecting mRNA levels, protein levels, or protein activity based on standard techniques well known in the arts. Detection may involve quantification of the level of gene expression (e.g., genomic DNA, cDNA, mRNA, protein, or enzyme activity), or, alternatively, may be a qualitative assessment of the level of gene expression, in particular in comparison with a control level.

In one example embodiment, detecting or determining expression levels of a Reg3A or KHSRP may include detecting or determining RNA levels for the Reg3A or KHSRP. For instance, one or more tissues from the subject may be obtained and RNA may be isolated from the said tissues. In a preferred embodiment, a tissue sample of brain, optic nerve, sciatic nerve, or spinal cord tissue may be obtained from the subject. RNA may be extracted from the tissue sample by methods well known in the arts. For instance, techniques for determining levels of gene expression include, but are not limited to, Northern analysis, RNase protection assays (RPA), microarrays and PCR-based techniques, such as quantitative PCR and differential display PCR.

In one example embodiment, activity or level of Reg3A or KHSRP protein may be measured prior to and/or subsequent to administering the agent disclosed herein to a subject. For instance, Reg3A or KHSRP protein expression levels may be assessed by any method well-known in the art may be detected and quantified by any of a number of means well-known to those of skill in the art. Aberrant levels of polypeptide expression of the polypeptides encoded by a Reg3A or KHSRP nucleic acid may be useful in determining a subject's response to being treated with an agent disclosed herein. Any method known in the art for detecting polypeptides may be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

Methods disclosed herein provide a competitive advantage by increasing growth capacity of the injured axons rather than simply blocking inhibitory signals. Additionally, methods disclosed herein may be complimentary or synergistic with the stem cell grafting. Current approaches for injury of brain and spinal cord axons revolve on providing stem cells to enhance recovery or using pharmacological means to overcome growth-inhibitory extracellular molecules by blocking receptor activation. In one example embodiment, administration of an agent disclosed herein may increase nerve regeneration by promoting axon growth in a subject. For instance, axon growth may occur in central nervous system (CNS) or peripheral nervous system (PNS) tissues of the subject.

In the central nervous system, extracellular factors increased by injury actively block axon regeneration after spinal cord, brain, and optic nerve injuries. Removal of KHSRP, which lies downstream of Reg3A signaling allows neurons to regrow axons on these non-permissive extracellular factors. Since both Reg3A and KHSRP are present in brain and spinal cord axons, utilizing methods herein to inhibit or deplete Reg3A signaling may improve regeneration after traumatic injury of the central nervous system.

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in an agent disclosed herein.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximate unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

The present disclosure may be better understood with reference to the following examples.

### EXAMPLES

### Example 1

### REG3A Protein Slows Axon Growth in Cultured Neurons.

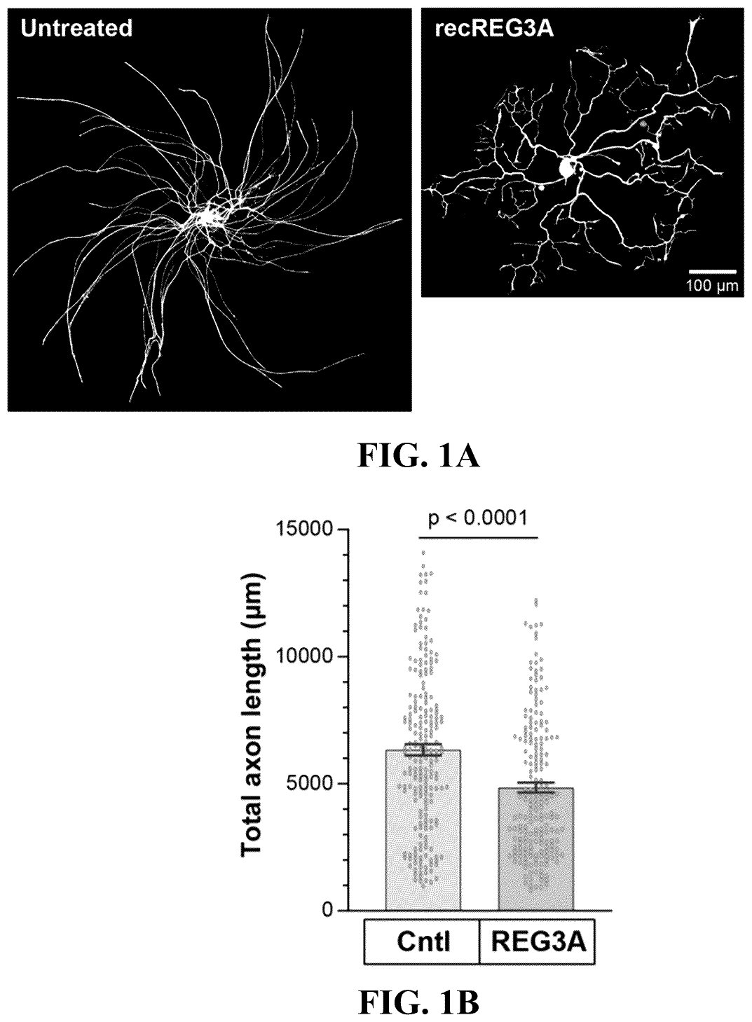

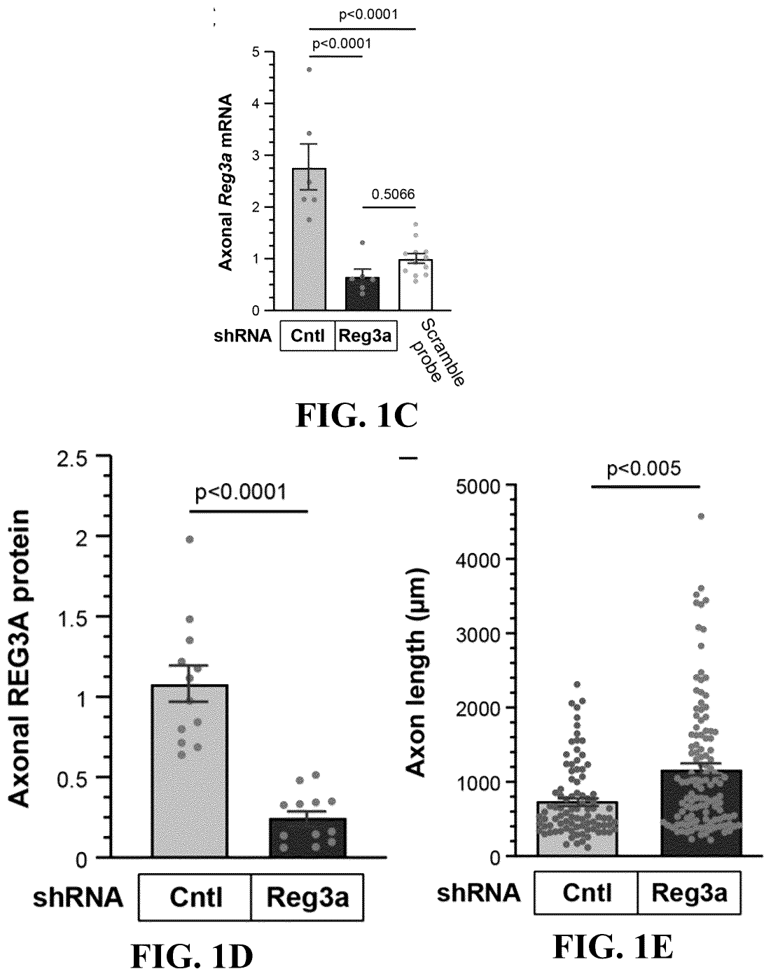

Previously, it was shown that Reg3A mRNA expression increases after nerve injury and the mRNA is transported into regenerating peripheral nerve axons and spinal cord axon. The spinal cord model used a peripheral nerve graft to support regenerating of ascending spinal cord axons after transection. To determine if REG3A protein might affect axon growth, whether the dorsal root ganglia (DRG) neurons respond to recombinant human REG3A (recREG3A) was investigated. Bath application of recREG3A reduced axon growth and increased axon branching in these adult mouse sensory neurons (FIGS. 1A-1B).

shRNA depletion of Reg3A mRNA was used to determine if the endogenous REG3A protein reduces axon growth. Adult mouse DRG neurons transfected with shRNA plasmid targeting Reg3a mRNA (shReg3a) showed reduced Reg3A mRNA and protein. This reduction in endogenous REG3A resulted in increased axon growth (FIG. 1E).

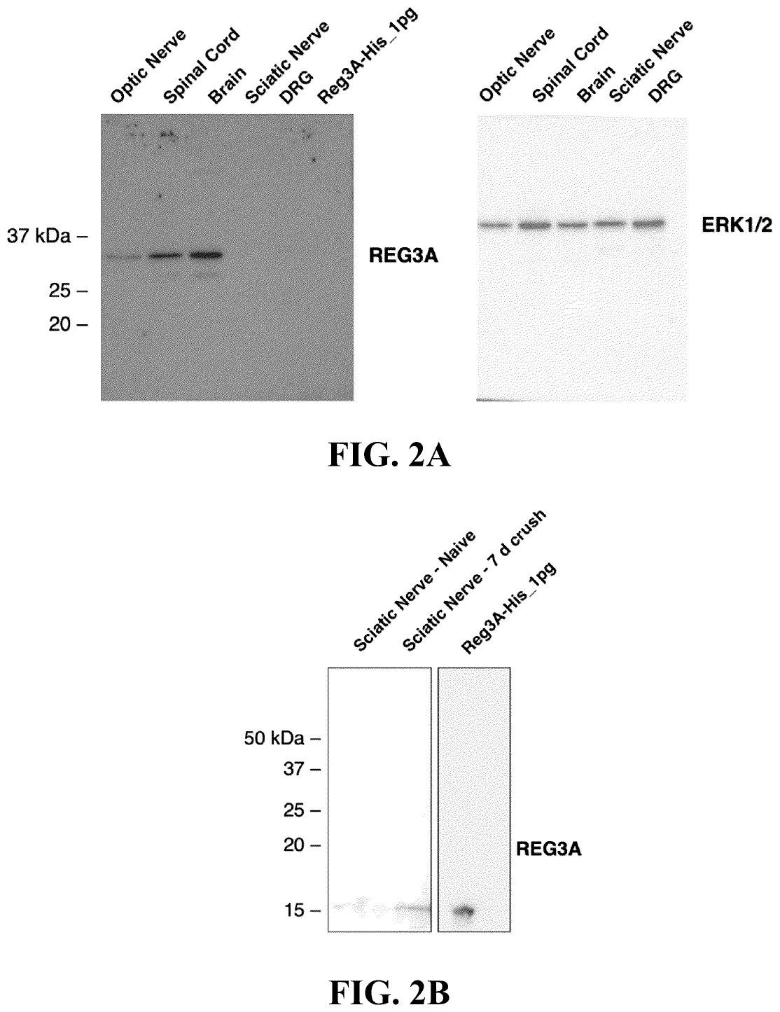

To determine expression levels of REG3A protein across the nervous system, initially immunoblotting was used for lysates of central nervous system (CNS) and PNS tissues. Protein bands recognized by anti-REG3A protein were clearly detected in adult uninjured brain, optic nerve, and spinal cord lysates but only very faint bands were seen in the uninjured sciatic nerve and DRG samples (FIG. 2A). These anti-REG3A detected bands unexpectedly migrated at approximately 30 kDa, while the predicted molecular weight of REG3A is about 15 kDa. Surprisingly, despite denaturation, dimers of REG3A protein were detected in these blots. Thus, a stronger denaturation procedure was used by boiling lysates in 4 M urea prior to electrophoresis. With this approach, a single band at approximately 15 kDa was seen in sciatic nerve lysates, with much higher levels in the 7-day crush injured compared to uninjured nerve (FIG. 2B).

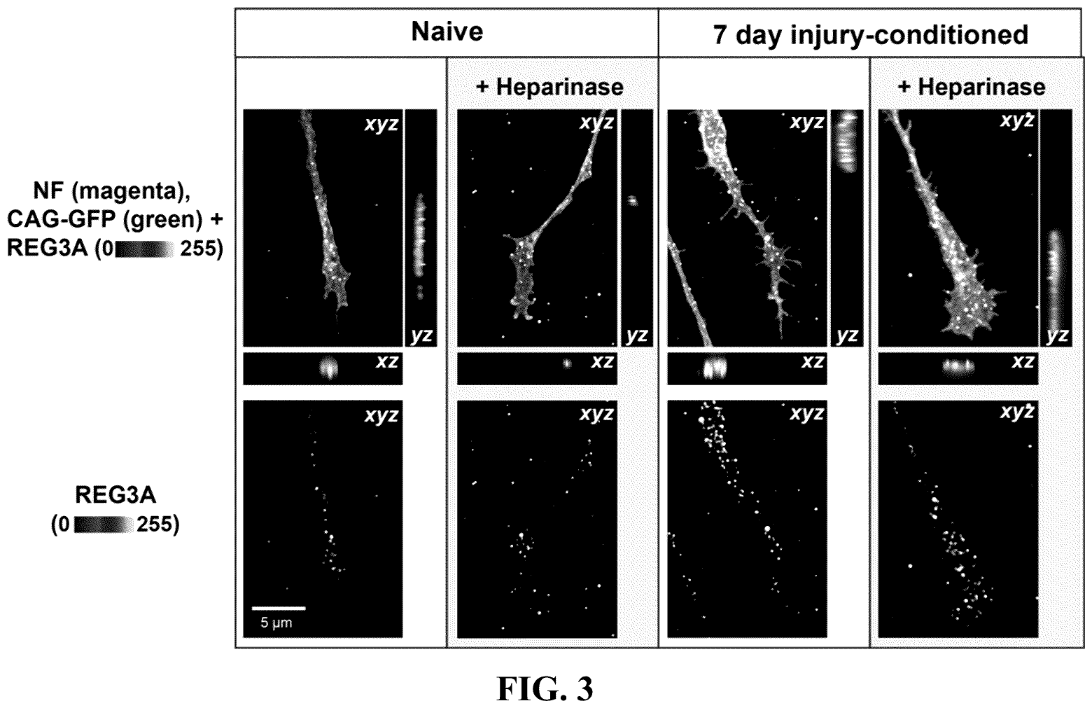

Further, an immunofluorescence was performed to visualize REG3A protein in DRG neurons cultured from naïve and 7-day sciatic nerve injury conditioned mice. There was a clear increase in overall REG3A immunofluorescence along axons for the neurons from the injury-conditioned mice (FIG. 3). Much of the REG3A immunosignals are along the periphery of the axons and there is a clear increase in immunoreactivity comparing the naïve and injury-conditioned DRG cultures (FIG. 3). REG3A has been reported to bind to carbohydrates and glycoproteins. Heparinase treatment prior to immunostaining showed depletes the signals along the axon, indicating that REG3A is ‘sticking’ to the surface of the axon (FIG. 3).

### Example 2

### REG3A Depletion Accelerates Peripheral Nerve Regeneration.

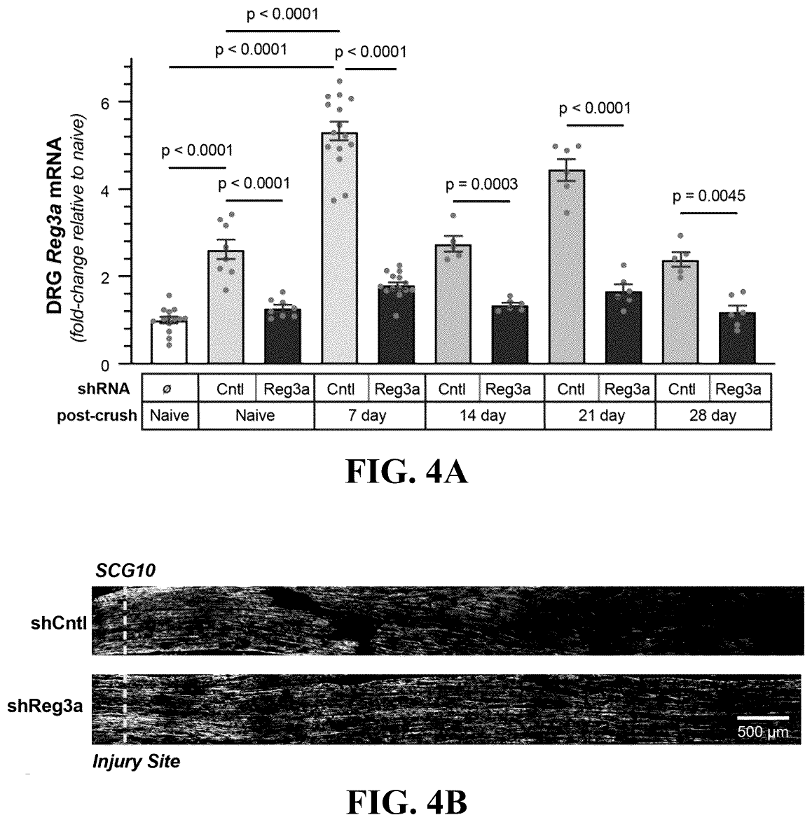

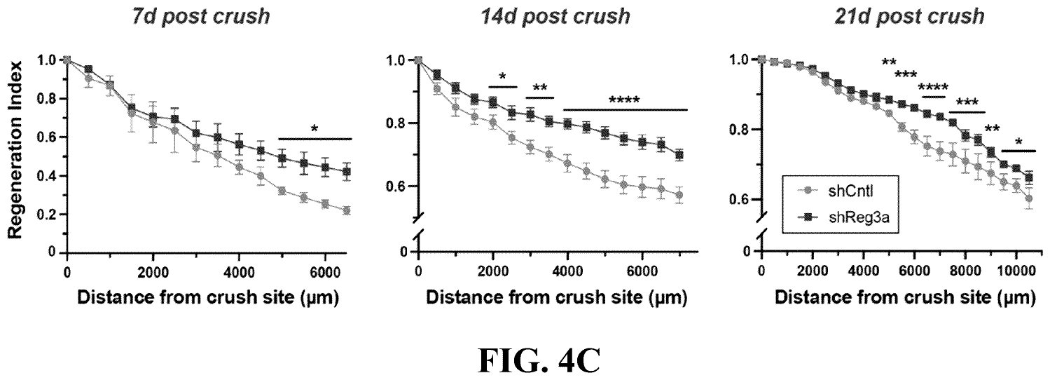

Given the effects of REG3A on axon growth in cultured neurons, whether the protein might also slow in vivo nerve regeneration was investigated. For this, neuronal REG3A was depleted by transducing with AAV-shReg3a vs. AAV-shCntl using direct injection into the sciatic nerve. AAV serotype is retrogradely transported to the neuronal cell bodies whose axons transverse the nerve resulting in a neuronal specific expression of the shReg3a and shCntl. Analyses of Reg3A mRNA levels showed a significant reduction of endogenous Reg3A mRNA the lumbar segment 4-6 (L4-6) DRGs whose axons traverse the sciatic nerve (FIG. 4A). Moreover, the AAV-shReg3a transduced mice showed increased axon regeneration at 7, 14, and 21 days post-crush injury (FIGS. 4B-4C).

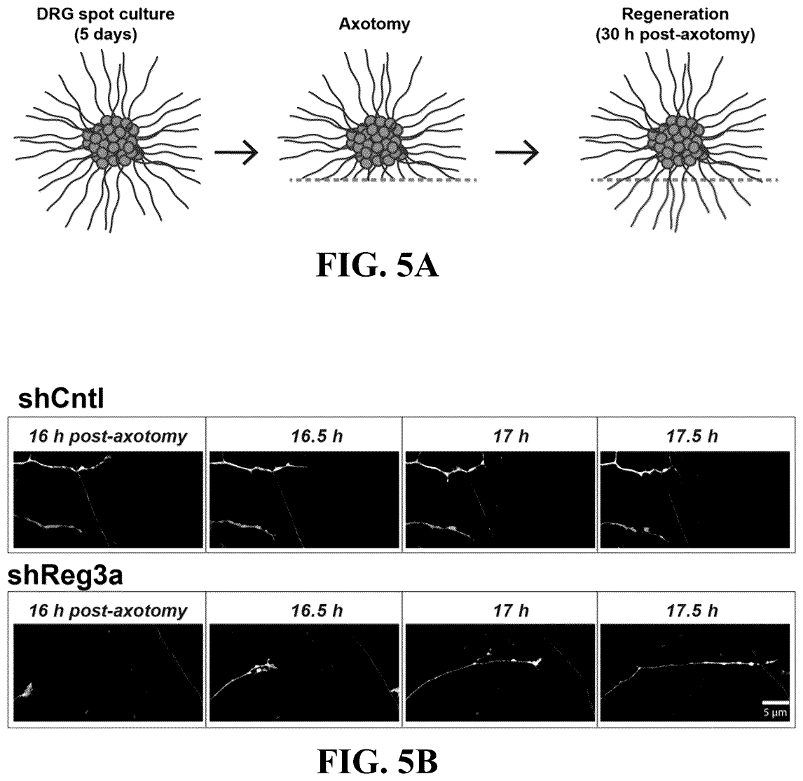

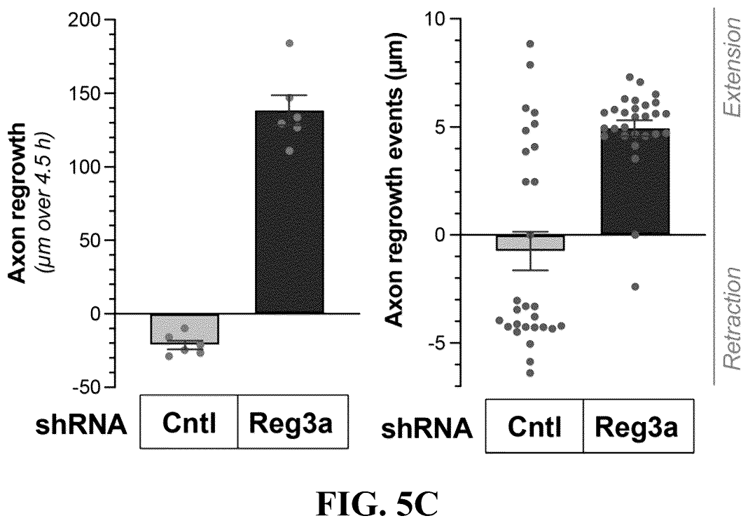

Next, whether endogenous REG3A slows the rate of axon regeneration was tested using a DRG ‘spot culture’ where the axon shaft could be severed and then the proximal axon monitored for axon growth by live cell imaging (FIG. 5A). Spot cultured neurons were transduced with AAV9-shReg3a or AAV9-shCntl, using virus preparations that also expressed blue fluorescent protein (BFP) for visualizing growing axons by live cell fluorescent microscopy. The AAV9-shReg3a transduced neurons showed faster axon growth than AAV9-shCntl transduced neurons (FIG. 5B). This was manifested in quantitation of overall growth across multiple cultures (FIG. 5C). Quantitating growth events across time points (i.e., the length an axon extended or retracted from one time point to the next) showed that the AAV9-shReg3a transduced neurons showed many more forward axon extension events than the AAV9-shCntl transduced neurons (FIG. 5C).

### Example 3

REG3A Activates the Translation of Axonal Khsrp mRNA.

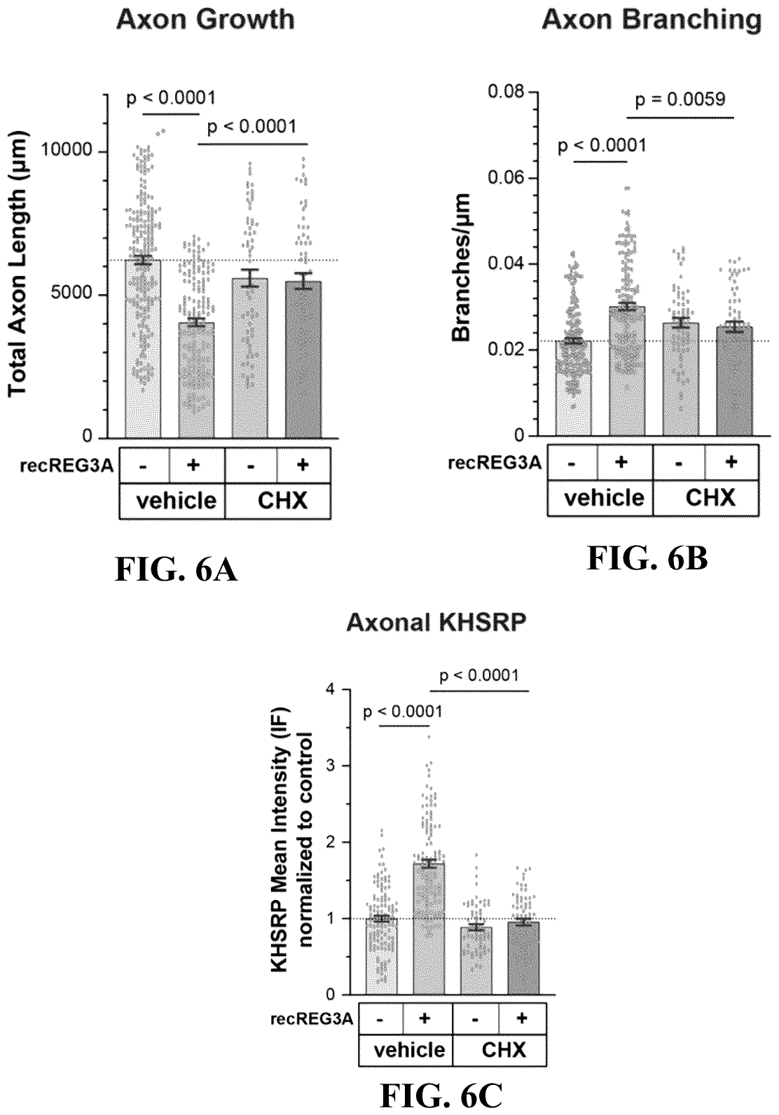

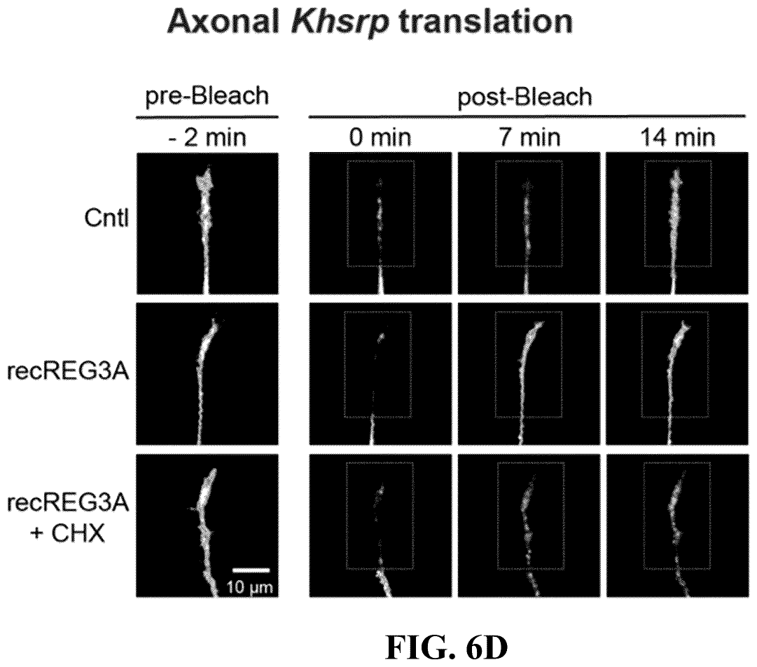

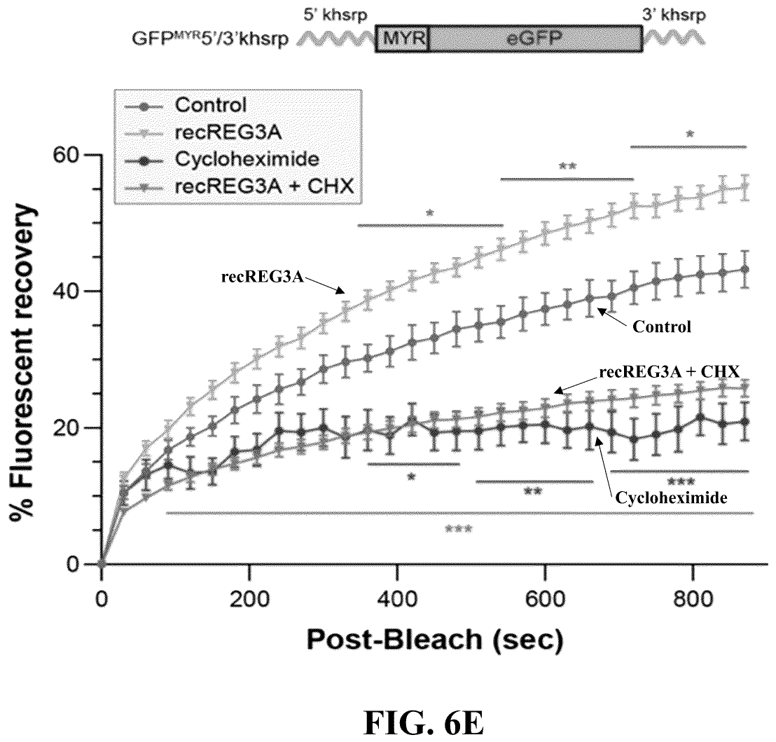

Previously, the accelerated nerve regeneration was observed with REG3A depletion mirrors in KHSRP knockout mice. Specifically, it was observed that KHSRP protein increases in regenerating sciatic nerve axons, which promotes decay of regeneration-associated mRNAs including Gap43 and Snap25 mRNAs. Thus, herein it was investigated whether REG3A effects might converge on Khsrp mRNA. The decrease in axon growth and increase in axon branching seen in dissociated DRG cultures treated with recREG3A was prevented by inhibition of mRNA translation using cycloheximide (FIGS. 6A-6B). recREG3A application also increased KHSRP levels in distal axons, but this did not occur in cultures where mRNA translation was inhibited with cycloheximide (FIG. 6C).

A fluorescence recovery after photobleaching (FRAP) assay was used to test whether REG3A can modulate translation of a reporter mRNA carrying the 5′ and 3′ untranslated regions (UTRs) of Khsrp mRNA (GFP^{MYR}5′/3′khsrp) as a proxy for intra-axonal translation of Khsrp mRNA (FIG. 6D). Diffusion limited reporters with myristoylation sequence (MYR) have consistently been used as surrogates for axon mRNA translation, and previously it was observed that fluorescent recovery of GFP^{MYR}5′/3′khsrp in axons is translation-dependent. recREG3A significantly increased fluorescent recovery of axonal GFP^{MYR}5′/3′khsrp signals compared to control conditions and this was attenuated by cycloheximide (FIGS. 6D-6E). Thus, REG3A effects converge on axonal Khsrp mRNA to increase its translation.

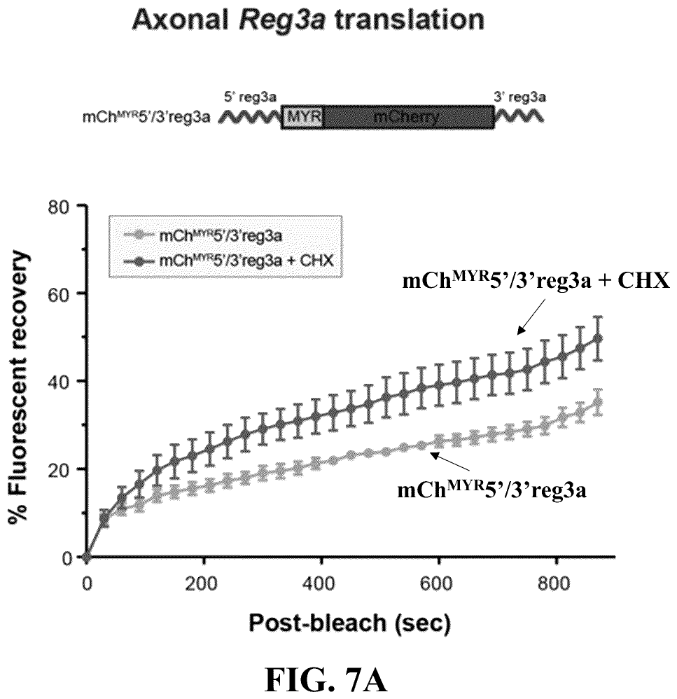

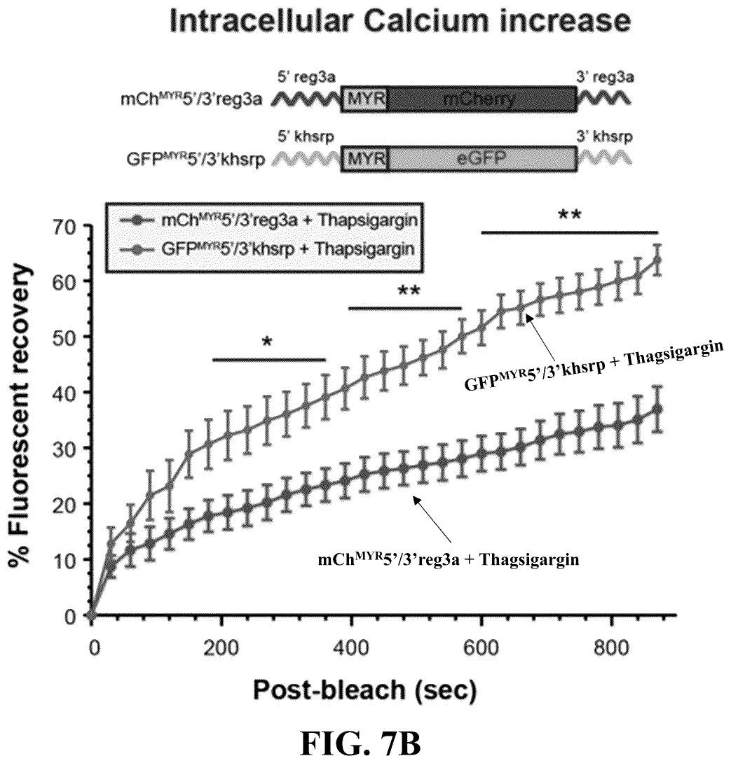

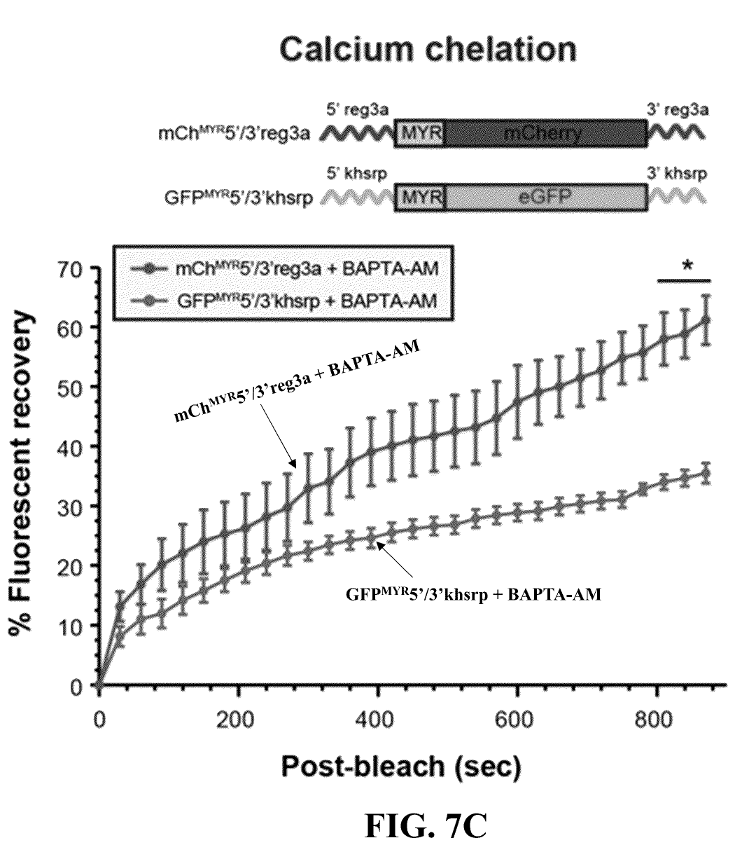

Khsrp mRNA translation is increased by elevated axoplasmic calcium and decreased by chelating intracellular calcium. Since Reg3a mRNA also localizes into the DRG axons, Reg3a mRNA response to modulation of axoplasmic calcium levels was investigated. By FRAP assay, mCherry^{MYR} with the 5′ and 3′ UTRs of murine Reg3a mRNA (mCherry^{MYR}5′/3′reg3a) showed recovery after photobleaching in transfected DRG neurons that was attenuated by protein synthesis inhibition (FIG. 7A). However, mCherry^{MYR}5′/3′reg3a and GFP^{MYR}5′/3′khsrp showed opposite response to calcium modulation in co-transfected neurons where axonal mCherry^{MYR} and GFP^{MYR} were simultaneously photobleached and monitored for fluorescence recovery. As previously published, increasing axoplasmic calcium by thapsigargin treatment, which blocks calcium uptake by the endoplasmic reticulum (ER), increased axonal GFP^{MYR}5′/3′khsrp translation but it decreased recovery of mCherry^{MYR}5′/3′reg3a (FIG. 7B). In contrast, chelation of intracellular calcium chelation with BAPTA-AM decreased axonal GFP^{MYR}5′/3′khsrp translation and increased axonal mCherry^{MYR}5′/3′reg3a translation (FIG. 7C). These data indicate that axonal synthesis of REG3A and KHSRP proteins s are increased under opposite physiological conditions with respect to axoplasmic calcium.

### Example 4

### REG3A Activates Release of Axonal Calcium Stores.

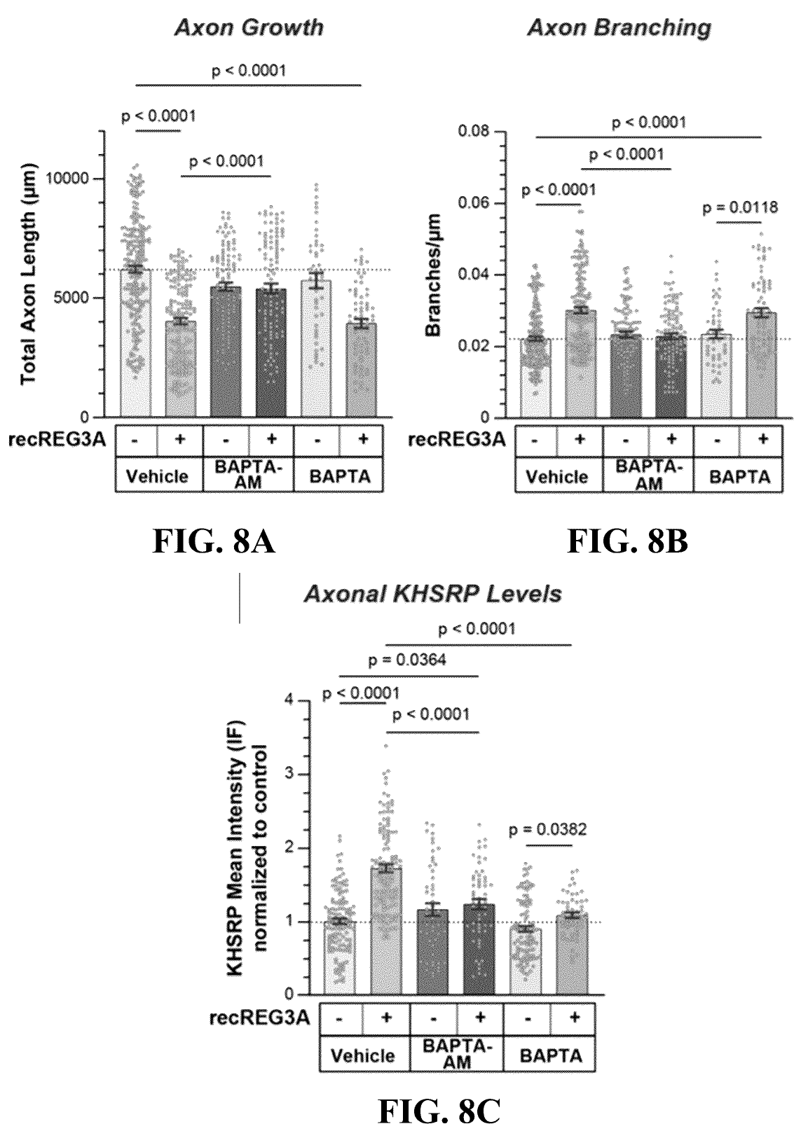

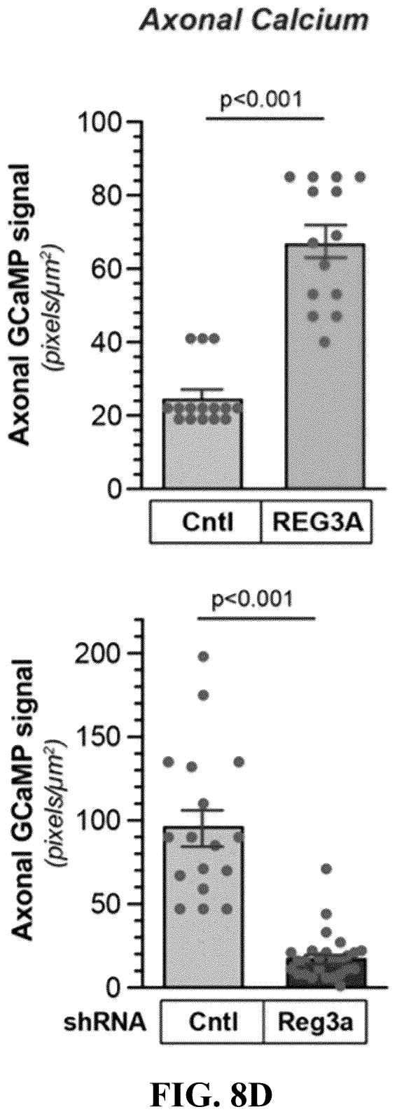

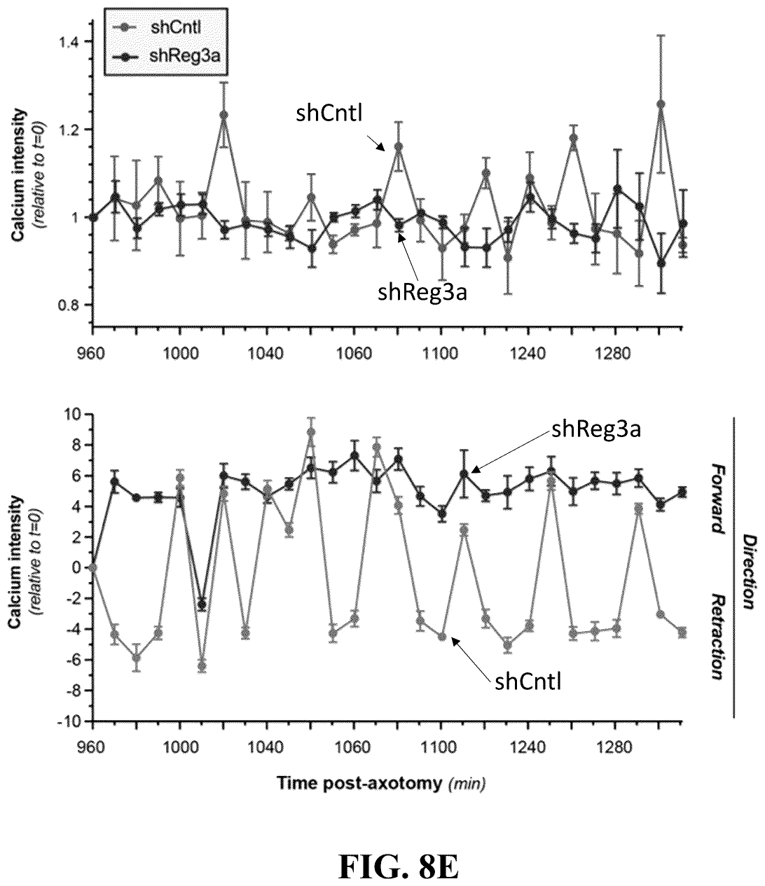

Since REG3A regulation of KHSRP synthesis requires increase in axoplasmic calcium, whether the calcium increase is from calcium entry into the axons (as seen initially following axotomy) or calcium release from intracellular stores was investigated. Chelating intracellular calcium using cell permeant BAPTA-AM but not extracellular calcium using non-cell permeant BAPTA blocked the effect recREG3A on axon growth and branching in dissociated adult mouse DRG cultures (FIGS. 8A-8B). The recREG3A-dependent increase in axonal KHSRP was attenuated by both BAPTA-AM, but BAPTA also attenuated the increase in axonal KHSRP protein (FIG. 8C), indicating that calcium entry from extracellular spaces can impact KHSRP synthesis in axons.

The findings above provide circumstantial but not direct evidence for elevated axoplasmic calcium as a key regulator of REG3A's effects on axons. As such, herein it was investigated whether REG3A increases calcium levels in DRG neurons using genetically encoded GCaMP fluorescent calcium indicator in DRG spot cultures. For this, DRG spot cultures were co-transduced with AAV9-GCaMP. Treating spot cultures with recREG3A significantly increased growth cone calcium (FIG. 8D). Neurons co-transduced with AAV9-GCaMP plus AAV9-shReg3a or AAV9-shCntl showed that depletion of endogenous REG3A significantly decreases growth cone calcium levels (FIG. 8D). Axonal calcium levels are known to increase immediately after axotomy but based on translational regulation of different axonal mRNAs that respond to elevated vs. normal axonal calcium levels, the axotomy induced increase in calcium returns to pre-injury levels within 16 hours after axotomy to allow translation of axonal mRNAs encoding regeneration-associated proteins. Thus, intra-axonal calcium levels in regenerating axons were monitored beginning at 16 hours after axotomy. The regenerating axons show an oscillatory GCaMP signal with broad peaks approximately every 50-70 min over a 4-hour period in AAV9-shCntl-transduced DRG spot cultures (FIG. 8E). These oscillatory calcium peaks in the axons were significantly blunted in cultures transduced with AAV9-shReg3a (FIG. 8E). Axon growth in the shCntl-transduced cultures showed retraction that roughly corresponded to or followed the peaks in calcium (FIG. 8E). In contrast, AAV9-shReg3a transduced cultures showed overall more processive axon growth events (FIG. 8E). These studies show that REG3A-induced changes in axonal calcium modulate axonal KHSRP levels and slow axon growth.

### Example 5

### REG3A Activates an Intrinsic Stress Response in Regenerating Axons.

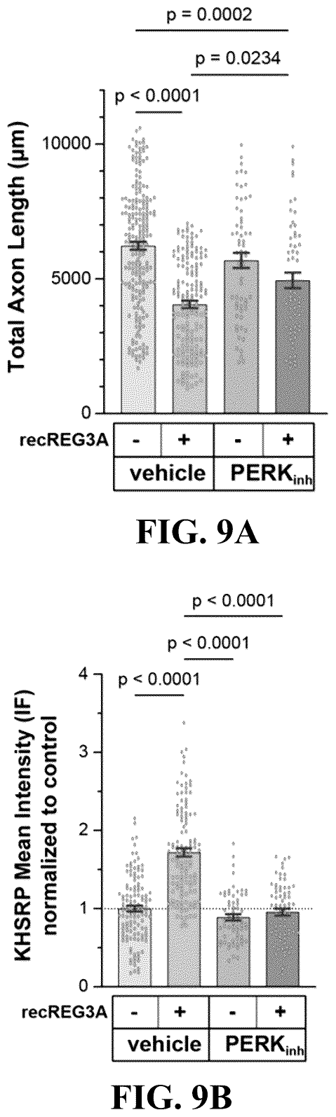

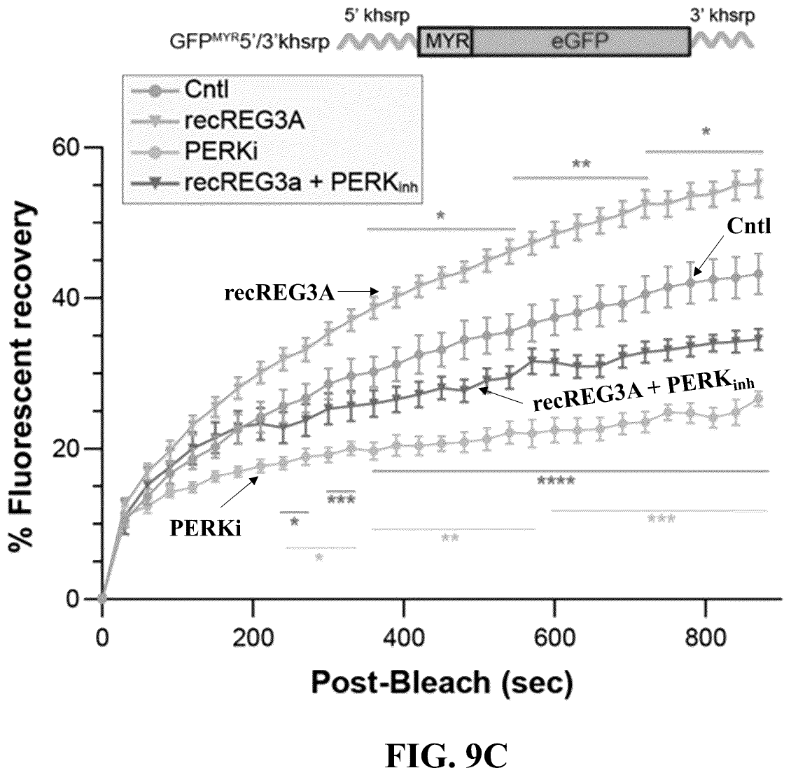

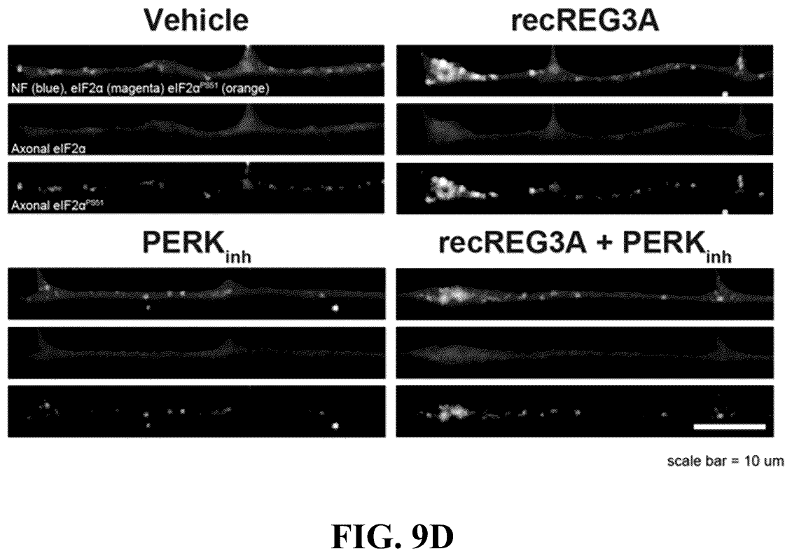

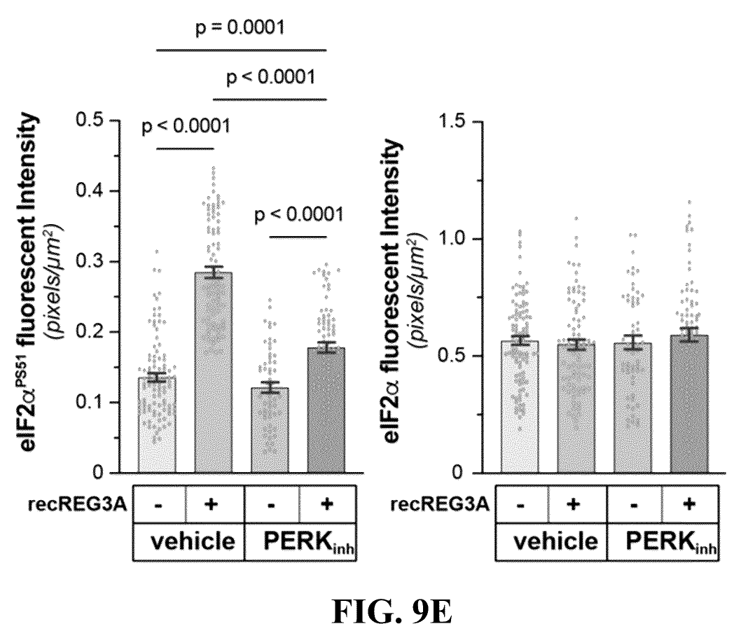

Previously, it was observed that axonal Khsrp mRNA translation is increased by activation of the PERK and subsequent PERK-dependent serine 51 phosphorylation of the translation initiation factor eIF2a. Though elevated eIF2α^{PS51} typically inhibits cap-dependent mRNA translation, translation of some mRNAs is paradoxically increased under that condition including several axonal mRNAs. Interestingly, it was observed herein that PERK inhibition attenuates REG3A's reduction in axon growth and elevation of axonal KHSRP (FIGS. 9A-9B). PERK inhibition similar reduced the REG3A-dependent translation of axonal GFP^{MYR}5′/3′khsrp in FRAP assays (FIG. 9C). Acutely treating adult mouse DRG cultures with recREG3A increased eIF2α^{PS51} levels in growth cones (FIGS. 9D-9E). Together, these data show that REG3A activates an intracellular signaling pathway that is known to regulate the translation of injury-response mRNAs including Khsrp mRNA.

### Example 6

Regenerating Axons Oscillate Between Translation of Reg3a and Khsrp mRNAs.

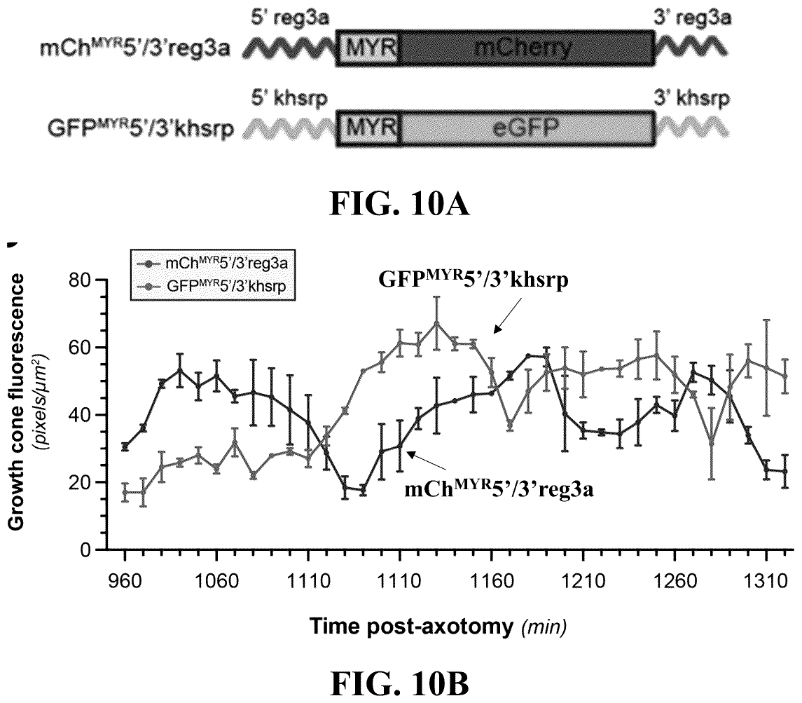

With the REG3A-dependent regulation of axonal Khsrp mRNA (FIG. 6), axonal translation of Reg3a mRNA (FIG. 7), REG3A-dependent oscillation of axoplasmic calcium levels (FIG. 8), and disparate regulation of Reg3a and Khsrp mRNA translation reporters with pharmacological calcium modulation (FIG. 7), whether regenerating axons might have reciprocally oscillating translation of Reg3a and Khsrp mRNAs was investigated. For this, axons of DRG spot cultures were transected that were co-expressing the mCherry^{MYR}5′/3′reg3a and GFP^{MYR}5′/3′khsrp translation reporters and quantified mCherry and GFP signals over time by live cell imaging (FIG. 10A). The axonal mCherry and GFP fluorescence oscillated over time with curves showing the opposite peaks and troughs-when mCherry was high in an axon, GFP was low and when GFP was high in an axon, mCherry was low (FIG. 10B). The periods of these peaks and troughs roughly correspond with the 50-70 min oscillation of axonal calcium levels and axon extension/retraction shown for the AAV9-shCntl transduced cultures shown in FIG. 8. This points to oscillating translation of axonal Reg3a and Khsrp that would result in overall slowing of axon regeneration.

### Example 7

Antisense oligonucleotides (ASO) have gained momentum as specific agents to prevent expression of individual gene products in the nervous system. Since Reg3a mRNA is an injury-induced gene, the mRNA offers oligonucleotide-based strategies for inhibition of REG3A protein expression in axons. Consistent with this, expressing a small hairpin RNA for the target sequence 5′-GATCCAACAATGGGTCAACAA-3′ (SEQ ID NO: 1) in the mouse Reg3a mRNA (Genbank #NM_011259.1) accelerates axon regeneration after traumatic nerve injury. This sequence corresponds to nucleotides 400-420, spanning across the junction of exons 4 and 5 in mouse Reg3a mRNA (Genbank #NM_011259.1). Intra-nerve injection of AAV serotype 5 encoding shRNA for this target sequence in mouse Reg3a mRNA is highly effective for depleting REG3A protein from injured neurons. Notably, the human REG3A gene has the same exonic structure as mouse REG3A gene. Thus, the region of mammalian Reg3a mRNA is an effective therapeutic target for intravenously-delivered Reg3a ASO to inhibit REG3A expression, including human (UniProt ID #Q13283), after axon injury and accelerate of regeneration.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method for increasing nerve regeneration, the method comprising:
administering an effective amount of an agent comprising a sequence at least 95% identical to SEQ ID NOs: 1-3 to a subject,
wherein the agent depletes or inhibits Reg3A or KHSRP expression in neurons of the subject.

2. The method of claim 1, wherein the agent is an RNA interfering agent.

3. The method of claim 2, wherein the RNA interfering agent comprises a small interfering RNA (siRNA), CRISPR RNA (crRNA), CRISPR guide RNA (gRNA), a small hairpin RNA (shRNA), a microRNA (miRNA), or a piwi-interacting RNA (piRNA).

4. The method of claim 3, wherein the RNA interfering agent comprises shRNA.

5. The method of claim 1, wherein the sequence is at least 97% identical to SEQ ID NO: 1.

6. The method of claim 1, wherein the sequence is at least 99% identical to SEQ ID NO: 1.

7. The method of claim 1, wherein the agent comprises SEQ ID NO: 1.

8. The method of claim 1, further comprising measuring a Reg3A or KHSRP expression level prior to administering the agent.

9. The method of claim 1, further comprising measuring a Reg3A or KHSRP expression level subsequent to administering the agent.

10. The method of claim 1, wherein the agent depletes or inhibits Reg3A or KHSRP expression by at least 70% or more subsequent to administering the agent as compared to a control sample.

11. The method of claim 10, wherein the agent depletes or inhibits Reg3A or KHSRP expression by at least 90% or more subsequent to administering the agent as compared to a control sample.

12. The method of claim 1, wherein the agent depletes Reg3A levels in neurons of the subject.

13. The method of claim 1, wherein administering the agent promotes axon growth at a nerve injury site in the subject.

14. A method for treating a nerve injury, comprising:
administering an effective amount of an agent comprising a sequence at least 95% identical to SEQ ID NOs: 1-3 to a nerve injury site of a subject,
wherein the agent depletes or inhibits Reg3A or KHSRP expression in neurons of the subject.

15. The method of claim 14, wherein the effective amount of the agent ranges from about 0.005 mg/kg to about 100 mg/kg.

16. The method of claim 15, wherein the effective amount of the agent ranges from about 0.01 mg/kg to about 30 mg/kg.

17. The method of claim 14, further comprising measuring a Reg3A or KHSRP expression level prior to administering the agent.

18. The method of claim 14, further comprising measuring a Reg3A or KHSRP expression level subsequent to administering the agent.

19. The method of claim 14, wherein the agent depletes or inhibits Reg3A or KHSRP expression by at least 70% or more subsequent to administering the agent as compared to a control sample.

20. The method of claim 19, wherein the agent depletes or inhibits Reg3A or KHSRP expression by at least 90% or more subsequent to administering the agent as compared to a control sample.


SEQUENCE LISTING (3 sequences)

SEQ ID NO: 1 - RNA, 21 nt, Mus musculus
  source 1..21: mol_type = genomic RNA
gatccaacaatgggtcaacaa

SEQ ID NO: 2 - protein, 7 aa, unidentified
DPTMGQQ

SEQ ID NO: 3 - protein, 7 aa, unidentified
DPTQGTE